(19) United States
(12) Patent Application Publication
(10) Pub. No.: US 2009/0035392 A1
(43) Pub. Date: Feb. 5, 2009

(54) User-adjustable treatment methods, systems and compositions for treating acne

(76) Inventor: Randall Wilkinson, Hayden, ID (US)

Correspondence Address: GRAYBEAL JACKSON LLP, 155 - 108TH AVENUE NE, SUITE 350, BELLEVUE, WA, 98004-5973, US

(21) Appl. No.: 11/450,881

(22) Filed: Jun. 9, 2006

Related U.S. Application Data
(60) Provisional application No. 60/732,785, filed on Nov. 1, 2005.

(51) Int. Cl. A61K 31/327; A61K 31/60; A61K 31/05; A61K 31/194; A61K 31/203; A61K 31/07; A61K 31/375; A61K 31/355; A61K 33/30; A61K 36/82; A61K 31/045; A61K 31/4166

(52) U.S. Cl. 424/642; 514/714; 514/159; 514/164; 514/731; 514/574; 514/557; 514/725; 514/474; 514/458; 424/729; 514/729; 514/390

(57) ABSTRACT

Methods, apparatus, systems and the like for treating acne vulgaris that improves treatment effectiveness and/or reduces bothersome side effects associated with the ingredients used. The present Methods, apparatus, systems, etc., also provide topical compositions of benzoyl peroxide and/or other anti-acne agents that are useful for treating acne vulgaris and may be used simultaneously or sequentially in a user-adjustable ratio of two or more such compositions, or other compositions as desired such as cleansers or lotions.

### CROSS-REFERENCE TO RELATED APPLICATIONS

The present application claims priority from U.S. provisional patent application No. 60/732,785, filed Nov. 1, 2005, which is incorporated herein by reference in their entirety and for all their teachings and disclosures.

### BACKGROUND

For an acne sufferer, one of its most vexing features is its variability. One day she can be feeling confident, attractive and comfortable. The next day her face can be marked by the red angry lesions, etc., of acne (Acne vulgaris) that can cause physical pain as well as leaving the sufferer feeling unattractive and less confident.

While many experience this rapid shifting between extremes, others find a gradual variation in severity. These variations typically occur without apparent cause, although for many females there can be a clear cyclic variability associated with their hormonal cycles.

Thus, there has gone unmet a need for improved methods, systems, etc., that enhance the ability of an acne sufferer to modify their treatment regimen at will so that increased acne can be met with increased treatment while decreased acne can be met with decreased treatment (and thus less harm to the skin).

Generally, there are four primary factors involved in the pathogenesis of acne.
1. Follicular hyperkeratosis and abnormal desquamation of follicular keratinocytes (generally, extra skin cell growth and shedding in the hair follicle). In the normal hair follicle, the movement of sebum in a film along the hair shaft toward the skin surface carries the desquamated debris out of the follicle. In acne, a thickened layer of superficial keratinocytes (hyperkeratosis) is released in clumps rather than as discrete or single cells (abnormal desquamation). This results in a plug of keratin debris and sebum forming at the neck of the follicle, obstructing the exit for sebum and keratin debris from the follicle.
2. Excess sebum production (sebum is an oily secretion of the sebaceous glands; with perspiration it moistens and protects the skin). As the sebocytes of the sebaceous gland are exposed to increased androgenic stimulus, increased amounts of sebum are secreted into the lumen of the follicle. With the exit channel blocked due to the hyperkeratosis and desquamation, there is a buildup of sebum within the follicle.
3. Proliferation of the acne-causing bacterium Propionibacterium acnes. P. acnes is a bacteria which is normally present in the thin film of sebum within the follicle. The buildup of sebum acts as a substrate for the proliferation of these bacteria within the follicle. While their increased presence within the follicle does not constitute an infectious disease, they secrete various inflammatory molecules which stimulate and perpetuate the local inflammatory response.
4. Immune and inflammatory responses. As a result of the initiation of the inflammatory process, a series of effects is caused. This includes, among others, the attraction of white blood cells into the area, tissue swelling and pain. This ongoing process can disrupt the follicular epithelium, resulting in the extravasation (exuding) of the built-up lipids, keratinocytes, bacterial antigens and inflammatory mediators into the surrounding dermis (skin). This process ultimately can lead to the painful cystic lesions and scarring associated with acne.

An effective treatment of acne preferably addresses each of these different causative factors. Multiple agents have been identified which affect one or more of these factors. Several have been approved by the FDA for use in the treatment of acne in over-the-counter (OTC) or prescription topical agents.

Benzoyl peroxide (BP), for instance, is a very effective treatment agent both for resolving the hyperkeratosis of the follicular wall and for decreasing the presence of P acnes. This effect is dose-related. That is, the higher the concentration of BP, the greater the keratolytic effect (helping to break up the plug) and the greater the antibacterial effect. However, although the increasing concentration of BP improves the factors leading to acne, it also increases the side effects associated with its use. This includes irritation of the skin, dryness, redness and peeling.

The result is that in formulating a treatment product for acne, a formulator typically balances the desire to increase the concentration of the active agent (to achieve better results) against the likelihood that the increased concentration will cause troublesome symptoms for the user.

The rising and falling occurrence of acne is such that, when the acne is flaring-up, a user may be willing to accept greater side effects than he or she would be when the acne is less active. But, when the acne symptoms have lessened, they may forego treatment because they are unwilling to accept the level of side effects associated with the high BP content of the product. Thus, the tendency of the severity of acne to change with time creates a problem in this trade-off between efficacy and side effects.

The picture is further complicated by individual susceptibility. With regard to side-effects, there is considerable variation from individual to individual in the tolerance their skin has to the negative effects of the treatment. The same concentration of BP that causes no problem to one individual may cause considerable side effects to another.

Further, individual susceptibility to side-effects is not a fixed feature. Rather, it can be subject to change under different conditions such as, for example, the tendency of some females to be more susceptible to side-effects at some times of the month compared to other times of the month due to hormonal changes associated with the menstrual cycle.

Individuality also impacts on the effectiveness of treatment aside from issues of side effects. The susceptibility to treatment of the bacterial strain colonizing one individual's follicles may be quite different from that of another individual. Similar dynamics may affect the susceptibility of one individual to the keratolytic effects of BP as compared to another individual. The result, again, is that the treatment concentration of BP which is sufficient for one individual may not be effective for another.

Thus, a set concentration of BP in an acne product may be problematic in achieving desired results. Various embodiments of the subject matter herein improves one or more of these issues.

### SUMMARY

In one aspect, the present methods, systems, apparatus, kits, etc., fill this need by providing for treating acne in a human by placing one or more acne medications in a dispensing vessel each at a predetermined concentration wherein the vessel is configured such that a patient can vary the concentration of at least one anti-acne agent in a composition dispensed from the vessel in accord with a desire of the user to treat acne. Examples of suitable acne medications are benzoyl peroxide, salicylic acid, sulfur, azelaic acid, resorcinol, resorcinol monoacetate, retinoic acid, retinol, ascorbic acid, alpha-hydroxy acids, and beta-hydroxy acids. The present methods, systems, apparatus, kits, etc., can further comprise dispensing at least one anti-acne agent to adjust the concentration of another anti-acne agent.

Addition exemplary components, with exemplary concentrations, include:
a. Antibiotics such as erythromycin (e.g., 2.0%); clindamycin (e.g., 1.0%), other topical antibiotics.
b. Retinoic Acids such as tretinoin (e.g., at 0.025% to0.1%).
c. Adapalene (e.g.,0.1%).
d. Tazarotene (e.g., 0.05%-1.0%).

The systems, etc., can further comprise simultaneously increasing a concentration of a first anti-acne agent in order to adjust the anti-acne effect of the anti-acne agent while decreasing a concentration of a second anti-acne agent in order to adjust the anti-acne effect of the anti-acne agent.

In another embodiment two, three, four or more ingredients are placed in separate vessels within a dispenser and each ingredient is dispensed and all of the ingredients are emitted together in a predetermined amount as desired by the patient for example using dispenser(s) based ink-jet technology.

One aspect herein can comprise methods of treating acne in a human comprising, providing a dispensing vessel containing one or more anti-acne agents wherein the vessel can be configured such that a patient can easily vary the concentration of at least one anti-acne agent in a composition dispensed from the vessel in accord with a desire of the user to treat acne. The anti-acne agent can be benzoyl peroxide, salicylic acid or other ingredients as desired. The concentration of the benzoyl peroxide can be between about 0% and 20% and the salicylic acid between about 0% and 10%, 1%and 5%, or 2.5% and 10%, and the salicylic acid can be between about 0.5% and 2%. The methods can further comprise the user adjusting the concentration of the at least one anti-acne agent in the composition dispensed from the vessel in order to adjust the anti-acne effect of the anti-acne agent, for example simultaneously increasing a concentration of a first anti-acne agent in order to adjust the anti-acne effect of the anti-acne agent while decreasing a concentration of a second anti-acne agent in order to adjust the anti-acne effect of the anti-acne agent, or simultaneously increasing at least two anti-acne agents, or independently adjusting a concentration of a first anti-acne agent in order to adjust the anti-acne effect of the anti-acne agent and independently adjusting a concentration of a second anti-acne agent in order to adjust the anti-acne effect of the anti-acne agent.

In some embodiments, the concentration of the benzoyl peroxide can be increased such that increased concentrations of the benzoyl peroxide can be administered when acne of the user is relatively worse and such that decreased concentrations of the benzoyl peroxide can be administered when acne of the user is relatively less. The methods can further comprise adjusting the concentration of the salicylic acid such that increased concentrations of the salicylic acid can be administered when acne of the user can be relatively worse and such that decreased concentrations of the salicylic acid can be administered when acne of the user is relatively less.

The first anti-acne agent can comprise at least one of resorcinol, azelaic acid, retinoic acid, retinol, ascorbic acid, alpha-hydroxy acids, beta-hydroxy acids and the second anti-acne agent can comprise at least one of resorcinol, azelaic acid, retinoic acid, retinol, ascorbic acid, alpha-hydroxy acids, beta-hydroxy acids. The first anti-acne agent and the second anti-acne agent can be selected such that the first anti-acne agent has at least one side effect that can be ameliorated by the second anti-acne agent. The first anti-acne agent and the second anti-acne agent can be selected such that at least one of the first anti-acne agent and the second anti-acne agent enhances the anti-acne effect of the other anti-acne agent.

The methods can further comprise administering the first anti-acne agent to the skin of the user during the evening and administering the second anti-acne agent to the skin of the user during the morning, and the methods can further comprise using at least two dispensing vessels, one for each of the anti-acne agents. The methods can further comprise administering at least one skin care active agent such as a tocotrienol, gorgonian, oleanolic acid, zinc, camellia sinensis, NDGA, bisabolol or allantoin.

Another aspect herein provides anti-acne systems comprising at least one dispensing vessel containing one or more anti-acne agents wherein the vessel can be configured such that a patient can easily vary the concentration of at least one anti-acne agent in a composition dispensed from the vessel in accord with the desires of the user. The systems can use the formulations discussed above and elsewhere herein. The anti-acne system can further comprise at least two anti-acne agents and the system can be configured such that the user can simultaneously increase, simultaneously decrease, inversely increase and decrease or independently increase/decrease a concentration of a first anti-acne agent while decreasing a concentration of a second anti-acne agent (or more).

The anti-acne systems can comprise at least a first compartment comprising a first sub-composition comprising the first anti-acne agent at a first concentration and a second compartment comprising a second sub-composition lacking the first anti-acne agent or comprising first anti-acne agent at a second, lower concentration, wherein the dispensing vessel can be configured such that the concentration of the at least one anti-acne agent can be increased or decreased by varying the relative amount of the first sub-composition and the second sub-composition dispensed from the dispensing vessel. The anti-acne systems can further comprise at least a third compartment comprising a third sub-composition, wherein the dispensing vessel can be configured such that the components of a composition dispensed from the dispensing vessel can be varied by varying the relative amount of the first sub-composition, the second sub-composition and the third sub-composition dispensed from the dispensing vessel. Such systems can comprise at least two, three or more different anti-acne compositions each contained in different compartments or at least some in the same compartment.

The anti-acne systems and methods, etc., can comprise a first anti-acne agent that has at least one side effect that can be ameliorated or enhanced by the second anti-acne agent. The first anti-acne agent can be an oxidizing agent and the second anti-acne agent can be an antioxidant, and the anti-acne systems can further comprise administering at least one skin care active agent. The anti-acne system can further comprise at least two dispensing vessels.

A further aspect herein provides methods of manufacturing a medicament able to reduce symptoms associated with acne in a human patient, comprising variably combining a pharmaceutically effective amount of at least one anti-acne agent and at least one carrier, adjuvant, excipient, potentiator or skin care active agent to provide a user-variable concentration of the anti-acne agent in an anti-acne composition comprising the at least one anti-acne agent and the at least one carrier, adjuvant, excipient, potentiator or skin care active agent. The methods can further comprise variably combining at least one additional anti-acne agent in the composition, or variably combining a pharmaceutically effective amount of at least one second anti-acne agent and at least one carrier, adjuvant, excipient, potentiator or skin care active agent to provide a user-variable concentration of the second anti-acne agent in a second anti-acne composition comprising the second anti-acne agent and the at least one carrier, adjuvant, excipient, potentiator or skin care active agent. The methods can further comprise placing at least one of the compositions in the hand of a user such that the user can administer the at least one of the compositions to skin of the user.

A still further aspect herein provides kits for providing compositions able to reduce symptoms associated with acne in a human patient, comprising at least one dispensing vessel as discussed herein and instructions for use according to the methods discussed herein. The kits can further comprise labeling approved by a governmental agency such as the FDA or non-US agency in non-US jurisdictions.

These and other aspects, features and embodiments are set forth within this application, including the following Detailed Description and attached drawings. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application.

### BRIEF DESCRIPTION OF THE DRAWINGS

FIG. 1 shows a variable-concentration dispenser system sold by Versadial Inc., New York, N.Y.

FIG. 2 shows the pump head of the variable-concentration dispenser sold by Versadial.

FIG. 3 demonstrates a ratio of 30:70 of ingredients being drawn through the Versidial pump.

FIG. 4 shows a diagram of a device suitable for use with multi-variate acne treatment systems.

FIG. 5 illustrates a device containing 3 acne medications having the approximate ratios of A:B:C are 1:1:1, with each component representing approximately 33% of the combined mixture being dispensed.

FIG. 6 is a front planar view of one embodiment of another exemplary dispenser.

FIG. 7 is a back view of the dispenser in FIG. 6.

FIG. 8 is a bottom view of the dispenser in FIG. 6.

FIG. 9 is an exploded view of the dispenser in FIG. 6.

FIG. 10 is a schematic cross-sectional view of a cartridge, flow path, and piezoelectric ink jet head.

FIG. 11 is a schematic cross-sectional view of a solenoid ink jet head.

FIG. 12 is a schematic cross-sectional view of a dual valve solenoid-piezo ink jet head system.

FIG. 13 is chart of exemplary benzoyl peroxide lotions suitable for use with the systems, methods, etc., herein.

FIG. 14 is chart of exemplary acne recovery lotions suitable for use with the systems, methods, etc., herein.

### DETAILED DESCRIPTION

An effective solution to this conflict is to provide the user with the ability to control the concentration of the active ingredient(s) in the acne treatment formulation. The user is thus able to respond to the unique demands of the severity of acne at a particular point in time as well as their own individual characteristics. In some embodiments, the user can simultaneously increase one active ingredient while decreasing another, or the user can independently increase and decrease the two or more ingredients. For example, the user can be provided with a vessel configured to adjust the relative concentration of a single active ingredient in a composition obtained from the vessel, or the user can be provided with one or more vessels configured to adjust the relative concentration of two different active ingredients in direct proportion with each other (both increase or both decrease at the same time), or the user can be provided with one or more vessels configured to adjust the relative concentration of two different active ingredients in inverse proportion with each other (one increases while the other decreases at the same time), or with one or more vessels configured such that the concentration of the different active ingredients are independently varied. The two or more active ingredients can be provided in a same composition or different compositions. The compositions can be in any desired form capable of variable concentration, for example lotions, gels, creams, liquids, solutions, suspensions, etc.

In some embodiments, the active ingredients are selected such that a first of the active ingredients is active against p. acnes and/or other acne causation factors due to oxidative or other effects, while a second active ingredient is selected such that it is active against p. acnes and/or other acne causation factors due to anti-oxidative or other effects that are also counteractive relative to the first active ingredient. Thus, in such a situation, it may be desirable to lower the concentration of one active ingredient relative to the other. It can also be advantageous to administer the two or more active ingredients at different times so that they don't counteract each other on the skin of the user, with possibly either negative side effects for the user and/or elimination of their respective beneficial effects. Moreover, the concentrations and time of delivery to the skin can be selected by the user to minimize publicly visible side effects. For example, an oxidizing, variable-concentration BP composition can be administered in the evening so that any redness, etc., occurs overnight while the user is sleeping, and then an antioxidizing, variable concentration salicylic acid composition can be administered in the morning (if desired, only one of the different compositions can be of a variable concentration configuration), which salicylic acid composition can both counteract the negative side effects of the BP while simultaneously clearing out the buildup of detritus in the hair follicles that traps the P. acnes in the follicle.

In another example using benzoyl peroxide, the user can be provided with the ability to select a concentration between, for example, 1% or 2.0% up to 5.0% or 10% w/w depending on his or her unique needs at that point in time. In certain embodiments, such an arrangement provides a concentration at, or above, the minimum concentration required by the FDA in a treatment composition, and at or below the maximum concentration allowed by the FDA in a treatment composition. Thus, when selecting the minimum concentration within the range, an adequate treatment dose is still being delivered. Yet, when a more effective dose is required, the user is able to immediately select the dose that best balances the need for efficacy and the need to minimize side effects.

This same approach can be used for any ingredient that might be used in an acne formulation that includes the balancing of efficacy and side effects, including where such balance can be affected by meeting individual needs. Some ingredients have been approved for use in acne treatment by the FDA in specified concentration ranges and are included within the current discussion. Those ranges may be redefined with time, and such redefined ranges are also included within the current discussion. Other ingredients have characteristics which may benefit acne but which are not currently approved by the FDA specifically for acne treatment although they may be in the future for acne treatment. Other anti-acne ingredients are also included within the current discussion.

Those in the following list are exemplary of those subject to the conflict between efficacy and side effects (the FDA minima and maxima are not confirmed for all the ingredients); the ranges below include all percentages (w/w) between the minima and maxima.

| | Exemplary | Exemplary | FDA | FDA |
|---|---|---|---|---|
| Ingredient | Low-Conc | High-Conc | Minimum | Maximum |
| Benzoyl Peroxide | 0.0% | 25.0% | 2.5% | 10.0% |
| Salicylic Acid | 0.0% | 25.0% | 0.5% | 2.0% |
| Resorcinol | 0.0% | 20.0% | 2.0% | 2.0% |
| Resorcinol | 0.0% | 20.0% | 3.0% | 3.0% |
| Monoacetate | | | | |
| Sulfur | 0.0% | 25.0% | 3.0% | 10.0% |
| Azelaic Acid | 0.0% | 25.0% | 10.0% | 20.0% |
| Retinoic Acid | 0.0% | 1.0% | 0.04% | 0.1% |
| Retinol | 0.0% | 25.0% | N/A | N/A |
| Ascorbic Acid | 0.0% | 25.0% | N/A | N/A |
| Alpha-hydroxy acids | 0.0% | 25.0% | N/A | N/A |
| Beta-hydroxy acids | 0.0% | 25.0% | N/A | N/A |

See: DiBemardino, et al. Cosmetic Bench Reference Directory of cosmetic ingredients 2005, Allured Publishing, 2005 (this and all other references are incorporated herein by reference in their entirety and for all their teachings and disclosures, regardless of where the references may appear in this application.).

In certain embodiments, the compositions comprising the one or more anti-acne ingredients include the additional skin care active agents such as tocotrienols, gorgonian, oleanolic acid, zinc, camellia sinensis, NDGA, bisabolol, allantoin and others as desired (note, such components may themselves also have some anti-acne activity and therefore can, in some embodiments, be selected as an anti-acne agent).

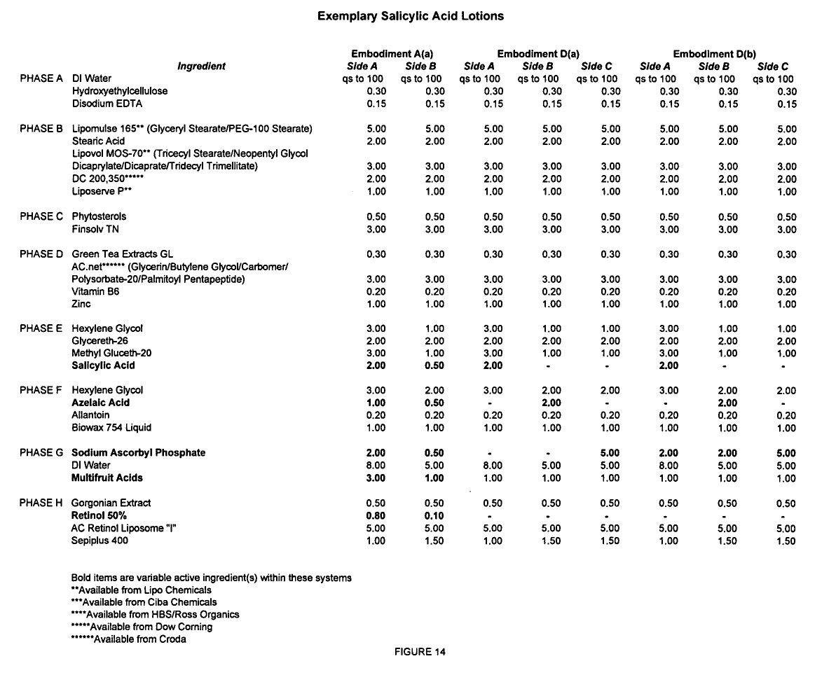

FIG. 13 provides a chart of exemplary benzoyl peroxide-containing lotion pairs suitable for use with the systems, methods, etc., herein. FIG. 14 provides chart of exemplary acne recovery lotion pairs suitable for use with the systems, methods, etc., herein. As with the other exemplary formulations herein, these examples are not limiting of the types and forms of components that can be included in the formulations herein. For example, The azelaic acid can be included in a reverse gradient such that the other actives decrease in concentration from vessel A to vessel B but the azelaic acid increases from A to B. One reason for such configuration is that azelaic acid may be incompatible with some of the other ingredients such as sodium ascorbyl phosphate, retinol, AC.net (a compound ingredient manufactured by Croda that includes oleanolic acid, NDGA and an osmotic antibacterial)). One example of this could be:
Embodiment A(a)—Side A: Azelaic Acid from 1.00 to 0.10
Embodiment A(a)—Side B: Azelaic Acid from 0.50 to 0.20

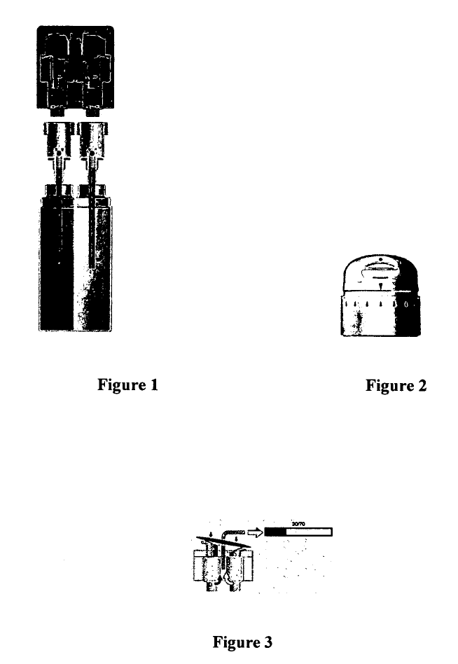

One example of how such a variable-concentration system may be implemented is a variable-concentration dispenser system by Versadial, http://www.versadialworld.com/, New York, N.Y. This dispenser system is configured such that a user can vary the ratio being drawn from two vessels and mixed prior to expulsion from a nozzle. This is illustrated in FIGS. 1-3.

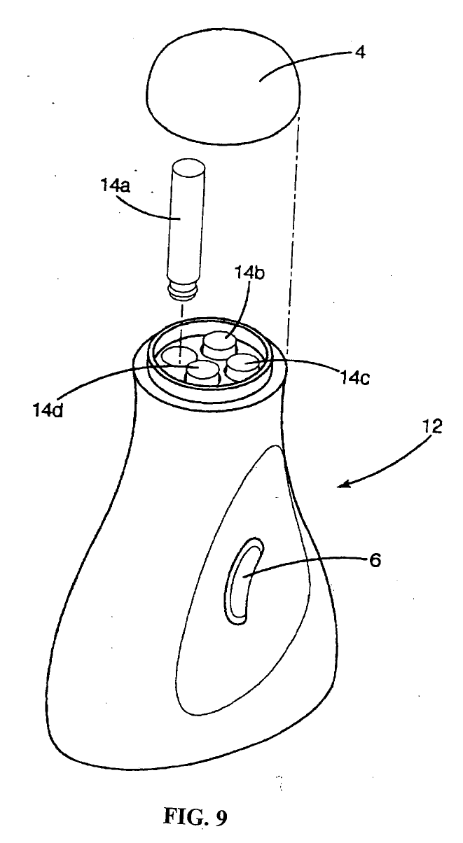

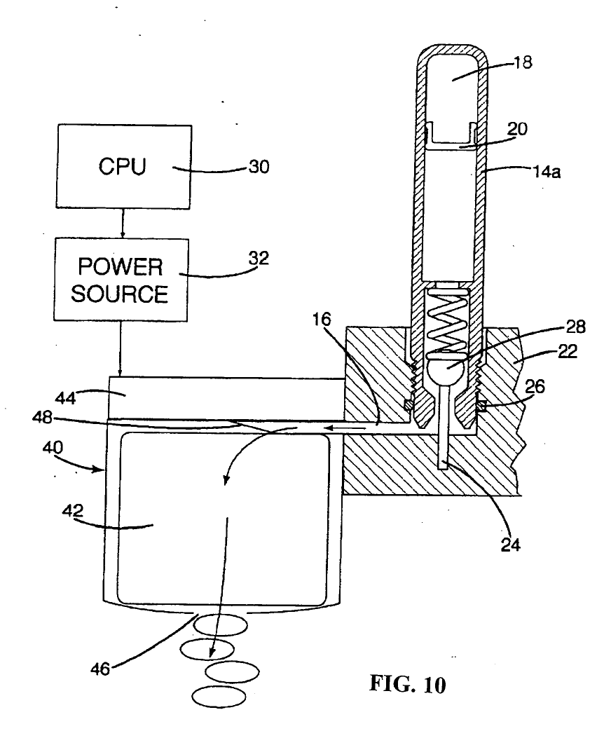

In this exemplary dispensing system as shown in FIG. 1, two vessels are used, 62 and 64. A pump 60 combines material drawn from each vessel, 62 and 64. Turning the pump head 66 of FIG. 9 varies the ratio of material drawn from each vessel. At one extreme of the head rotation, 100% of the expelled material is drawn from vessel 62 and 0% from vessel 64. As the head is rotated, the ratio gradually transitions from 100:0 to 90:10, 80:20, etc. At the other extreme of the rotation, 0% is drawn from Vessel 62 and 100% is drawn from Vessel 64. FIG. 10 demonstrates a ratio of 30:70 being drawn through the pump. If desired, the dispensing systems can be configured such that at least some amount of sub-composition is always taken up from one or more of the vessels. For example, the head may rotate between a 90:10 to 10:90 contribution from each of the vessels holding the sub-compositions.

The following are some exemplary embodiments of useful compositions, systems, methods, etc.
A.) Single Variable Ingredient (Unidirectional). In these embodiments, there are two (or more) vessels from which the user adjusts the ratio released of the given anti-acne active ingredient. There is one anti-acne active ingredient that is either absent in the lower-concentration vessel or at a specified concentration that is lower than the concentration in the higher-concentration vessel. Conversely, there is a higher concentration of anti-acne active ingredient in the higher-concentration vessel. The result is a single ingredient that increases from a zero or low concentration to a higher concentration as the ratio is adjusted between the two vessels.
   a. Exemplary Zero—Max spread: 0.0%-2.0%
   b. Exemplary Min—Max spread: 0.2%-2.0%
B.) Multiple Variable Ingredients (Unidirectional, parallel). In these embodiments, there are two or more vessels from which the user adjusts the ratio released. There is more than one ingredient that varies as the ratio from each vessel is adjusted. Each ingredient moves in parallel with an increasing concentration as the ratio is adjusted, from a zero concentration starting point for each, a minimum concentration starting point for each or a combination of zero and minimum concentration starting points.
   a. Exemplary Zero—Max spread: Compound A at 0.0%-2.0%; Compound B at 0.0%-10.0%.
   b. Exemplary Min—Max spread: Compound A at 0.5%-2.0%); Compound B at 2.0%-10.0%.
   c. Exemplary Zero—Max and Min—Max spread: Compound A at 0.0%-2.0%; Compound B at 2.0%-10.0%.
C.) Multiple Variable Ingredients (Bi-directional). In these embodiments, there are two or more vessels from which the user adusts the ratio released. There is more than one ingredient that varies as the ratio from each vessel is adjusted. One or more ingredients moves in an increasing concentration as one or more other ingredients moves in a decreasing concentration as the ratio released from each vessel is adjusted.
   a. Exemplary Zero—Max and Max—Zero spread: Compound A at 0.0%-2.0%; Compound B at 10.0%-0.0%.
   b. Exemplary Min—Max and Max—Min spread: Compound A at 0.5%-2.0%; Compound B at 10.0%-0.1%.
   c. Exemplary Zero—Max and Max—Min spread: Compound A at 0.0%-2.0%; Compound B at 10.0%-5.0%.

The examples above are not limiting. For example, various combinations and permutations of the embodiments above can also be applied to systems, methods, etc., having two or more variable ingredients. For example, there can be multiple variable ingredients in either a Uni-directional set-up or a Multi-directional set-up. There can also be more than two vessels from which ingredients are drawn in a manner allowing the user to determine the relative ratios drawn from each vessel.
d. Exemplary Zero—Max, Zero—Max, Zero—Max: Compound A at 0.0%-2.0%), Compound B at 0.0%-5.0%, and Compound C at 0.0%-10.0%).
e. Exemplary Zero—Max, Zero—Max, Min—Max: Compound A at 0.0%-2.0%, Compound B at 0.0%-5.0%, and Compound C at 5.0%-10.0%)
f. Exemplary Zero—Max, Max—Zero, Max—Zero: Compound A at 0.0%-2.0%, Compound B at 10.0%-0.0%, Compound C at 5.0%-0.0%)
g. Exemplary Zero—Max, Max—Zero, constant: Compound A at 0.0%-2.0%, Compound B at 10.0%-0.0%, Compound C at 5.0%-5.0%)

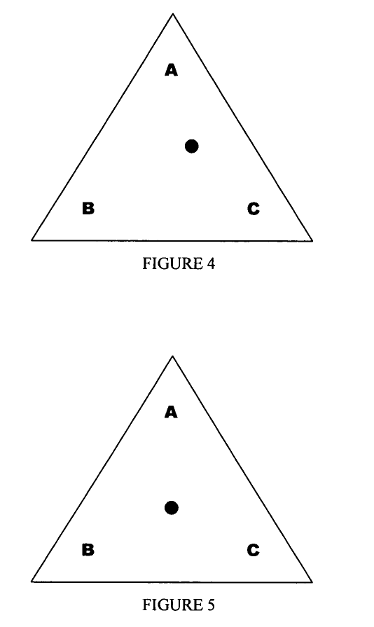

Turning to another exemplary embodiment, a device suitable for use with such multi-variate systems can be configured as shown in FIG. 4. In this dispensing system, a “joystick” is used to select the balance of material to be drawn from each of the three vessels of the system. In the exemplary figure shown above, this type of dispensing system is demonstrated for dispensing of materials under section “D” above. The location of the “joystick” dot indicates that an approximate ratio of A:B:C would be 5:1:5, with the three components A, B and C representing approximately 45%, 10%, 45%, respectively, of the combined mixture being dispensed.

In FIG. 5, the approximate ratios of A:B:C are 1:1:1, with each component representing approximately 33% of the combined mixture being dispensed.

In one embodiment, the three or more ingredient system can comprise three different vessels each containing one of the desired active ingredients, and then a variably sized pick up tube is operably connected to each one; as the joystick is moved, the size of the pick up tube is varied accordingly. In another embodiment, each of the desired ingredients is represented by a plurality of vessels each (if desired) having a different concentration of the desired ingredient and the system is configured such that moving the joystick moves the pick-up tube from one supply vessel to another so that the relative ratio of each of the ingredients is varied accordingly.

The compositions can have any other desired components suitable for use in a dermatological composition, which may comprise a sterile aqueous or non-aqueous solution, suspension or emulsion. Such additional components can comprise, for example, modulating agents, a dermatologically acceptable carrier (e.g., a non-toxic material that does not interfere with the activity of the active ingredient(s)), binder, excipient, buffer, adjuvant, dispersion agent, or other desired element. Any suitable carrier, etc., may be employed in the dermatological compositions. Representative carriers may include physiological saline solutions, gelatin, water, alcohols, natural or synthetic oils, saccharide solutions, glycols, organic esters such as ethyl oleate or a combination of such materials or other materials. Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, antimicrobial compounds, chelating agents such as EDTA or glutathione, adjuvants (e.g., aluminum hydroxide), inert gases or preservatives. Dermatological compositions may also contain other compounds, which may be biologically or therapeutically active or inactive. The compositions may be administered as part of a sustained release formulation (e.g., a formulation such as microcapsules that effects a slow release of compound following administration). Such formulations may generally be prepared using well known technology or otherwise. Sustained-release formulations may contain the active ingredient(s) dispersed in a carrier matrix or contained within a reservoir surrounded by a rate controlling membrane. Carriers for use within such formulations are biocompatible, and may also be biodegradable. In some embodiments, the formulation provides a relatively constant level of release.

In exemplary formulations, the compositions can comprise a) a safe and effective amount of an anti-acne agent such as benzoyl peroxide, salicylic acid, resorcinol, resorcinol monoacetate, sulfur, azelaic acid, retinoids including retinoic acid, alpha-hydroxy acids, beta-hydroxy acids, retinol, and ascorbic acid, as well as derivatives and mixtures thereof; b) a safe and effective amount of at least one additional skin care active ingredient, such as desquamatory actives, anti-acne actives, retinoids, hydroxy acids, liposomes, antioxidants, anti-seborrheics, 5-alpha reductase inhibitors, clays, keratolytics, exfoliants, melanogenesis inhibitors, radical scavengers, chelators, anti-inflammatory agents, topical anesthetics, skin lightening agents, flavonoids, antimicrobial actives, skin healing agents, allantoin, glucosamine, feverfew, aloe barbadensis, arnica montana, bisabolol, chamomile, coneflower, lecithin, gorgonian, guaiazulene, oleanolic acid, phytosterols, panthenol, salix alba, meadowsweet, phytosterols, vitamin B₆, zinc, camellia sinensis, quercitin, hydroquinone, kojic acid, coleus oil, fireweed, melaleuca alternifolia, niacinamide, Oregon grape root extract, tetrahydropiperine, tocopherol, tocotrienol, grape seed, rosemary, witch hazel, rosa canina, nordihydroguaiaretic acid (NDGA), magnesium, black cohosh, St. John's Wort, ethyl lactate, perfluorodecalin, pyridoxine dioctenoate, pyridoxine dipalmitate, laminaria saccharina, as well as derivatives and mixtures thereof; and, if desired c) an additional pharmaceutically acceptable dermatologic carrier. Other ingredients and combinations of ingredients are also possible.

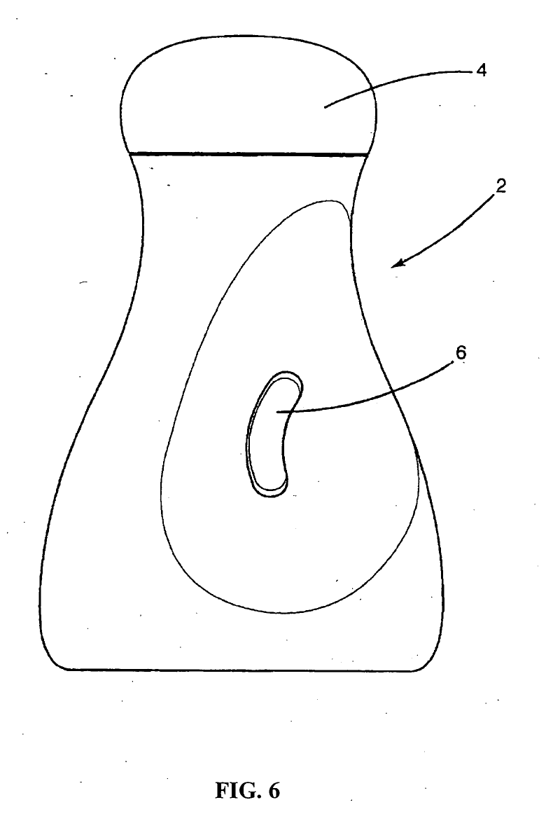

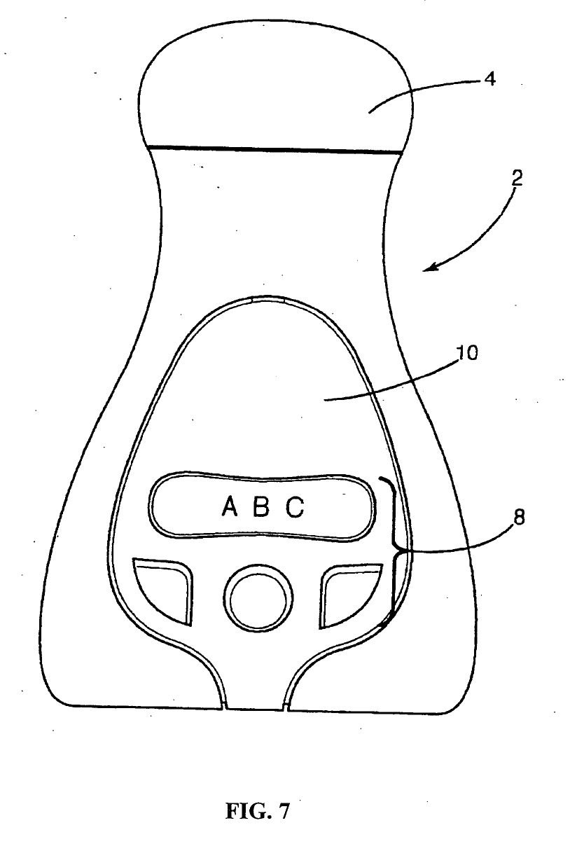

Another exemplary device that can be used to store, administer, etc., acne compositions as discussed herein is set forth in U.S. Pat. No. 6,715,642 as shown in FIGS. 6-12. This exemplary device uses ink jet printing technology to dispense a variety of acne compositions (liquid, gel, etc.). The ink jet head may use a magneto-restrictive alloy, thermal, solenoid, or piezoelectric technology. An exemplary piezoelectric system for custom formulating compositions will be described. Piezoelectric technology uses piezo crystals which receive a tiny electric charge causing the crystals to vibrate. At one instance, the crystal pulls back to allow fluid into the reservoir. At another instance, the crystal fires back into its original position exerting a mechanical pressure on the fluid which forces a tiny amount of fluid out of the nozzle. The typical ink jet head forces out small droplets of fluid, generally between 50 to 60 microns in diameter.

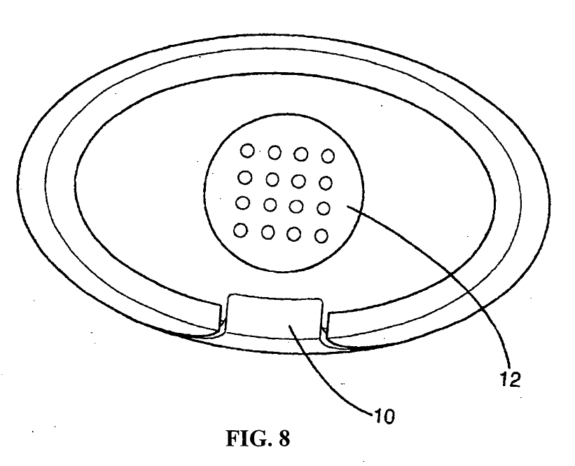

FIG. 6 provides side view of the exterior of a suitable dispenser2 comprising a lid 4 atop a body having a cover with a button 6. In FIG. 7 the multi-chambered dispenser 2 include several buttons that function to increase or decrease the amount of liquid that is dispensed from the cartridges 14a-14d. A removable cover or door 10 may partially or wholly cover the control panel 8. FIG. 8 shows the bottom view of the dispenser 2 showing a dispensing port 12.

Referring to FIG. 9, the exemplary multi-chambered dispenser 2 houses four cartridges 14a, 14b, 14c, 14d that each contain different anti-acne compositions, recovery compositions, etc., as desired. Each cartridge 14a-14d may hold about 1 ml to about 15 ml of liquid or gel composition. The cartridges 14a-14d are pressurized so the composition contained therein can easily pass out of the cartridges 14a-14d and into its corresponding flow path 16 shown in FIG. 10.

FIG. 10 is a schematic drawing of a piezoelectric system showing only one cartridge 14a and corresponding flow path 16 and a piezoelectric ink jet head 40. Although the four cartridges 14a-14d in FIG. 9 are not shown, this schematic drawing generally applies to each cartridge 14a-14d (or more if desired). Each flow path 16 empties into a corresponding chamber 42. The cartridges 14a may also include a plunger 20 for assisting in dispensing liquid from the cartridge to the flow path 16. In one embodiment, pressurized gas is disposed in a compartment 18 behind the plunger 20 to apply a force to the plunger 20. In some applications, the pressurized gas can be replaced by a spring or other conventional biasing mechanism, or manual devices. Alternatively, the cartridge 14a may use capillary action to move the composition into the ink jet head 40. The cartridge receiving end 22 of the flow path 16 may include a rod shaped plug 24 that breaks the cartridge seal when the cartridge 14a is coupled to the receiving end 22 of the flow path 16 as well as an o-ring 26. O-ring 26 surrounds the outside of the cartridge to prevent the liquid from leaking out around the edge of the cartridge 14a. The seal may be a spring-loaded ball 28 as shown in FIG. 10, a conventional foil seal, or natural surface tension. The cartridge 14a may be threaded or otherwise coupled to the receiving end 22 of the flow path 16.

In another embodiment of the this device, one cartridge 14a may feed into multiple ink jet heads 40. For example, each cartridge 14a-14d might have three flow paths 16, each leading into a separate ink jet head 40 (not shown). These multiple ink jet heads 40 are configured such that the different liquids are interlaced.

Still referring to FIG. 10, the control panel 8 is used to input a formula comprising a ratio of each foundation from the cartridge, the formula is received by a microprocessor (“CPU”) 30. The CPU 30 processes the inputted information and controls the amount of power generated from the power source 32 in activating the ink jet head 40. Fluid in the chamber 42 of the ink jet head 40 is subsequently dispelled by a change in the momentum of a momentum transferring device such as a piezo crystal 44 which is opposite the orifice 46 of the ink jet head 40. This abrupt change in momentum is conferred to the static liquid within the chamber 42 causing it to assume this momentum and propel from the orifice 46. A typical orifice of an ink jet head is about 0.002 inches in diameter. The orifice 46 of an ink jet head for dispensing composition can be about 0.007 inches to about 0.008 inches in diameter. Further, due to the rheology of composition, more than one momentum transferring device can be incorporated to assist in propelling fluid out of the chamber 42. This momentum can be conferred by a thermal system, solenoid actuator, piezo crystal or magneto-restrictive alloy. Any combination of the aforementioned momentum transferring devices or other devices as desired can be employed.

FIG. 10 is a piezoelectric ink jet head 40 and uses a piezo crystal 44. The ink jet head 40 includes a piezo crystal 44 that reacts to an electrical impulse communicated through the CPU 30 by the power source 32. When the piezo crystal 44 receives the electrical impulse, the impulse reconfigures the piezo crystal 44. The continual reconfiguration results in the piezo crystal 44 oscillating up and down. The piezo crystal 44 may oscillate at about 2,000 Hertz via electrical impulse from the power source 32. The liquid enters the ink jet head through a one way path on the uppermost layer of the piezo crystal 44. A flexible film 48 may be provided near the entry of the chamber 42 of the ink jet head 40 to assist in controlling the flow of composition through the flow path 16 and chamber 42 until it reaches the orifice 46. The force of the piezo crystal 44 while oscillating in a downward direction assists in transferring the composition out the orifice 46 of the ink jet head 40. The piezo crystal 44 in this embodiment acts as the momentum transferring device.

Because the fluid is not being actively pumped from a nozzle, measuring the quantity of dispensed fluid is typically not achieved by using a flow meter. Rather, in one embodiment, metering can rely on a calculation of the volume of the chamber 42 in relation to the number of times it is struck by the momentum transferring device. Some work may go into making sure that liquids of varying rheology consistently dispense with a fixed volume. Once this volume is known, one can achieve a desired ratio of liquids simply by controlling the oscillations of the momentum transferring device.

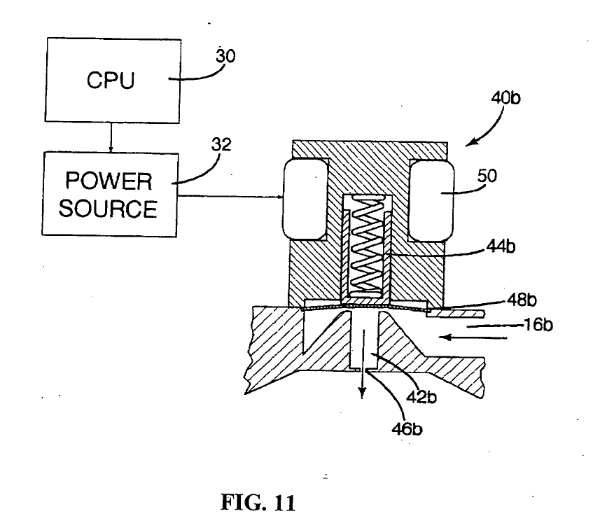

The composition may dispense from the orifice 46 in the form of spherical droplets of finite volume. In one embodiment, there are approximately 50,000 drops that total approximately 0.1 ml for each cycle or for each time a user activates the dispenser. Droplet size may vary from application to application depending on the characteristics of the ink jet head (e.g. ink jet orifice diameter) and the dispensed liquid (e.g. rheology and viscosity). Other types of ink jet head systems may be employed in such a device. FIG. 11 shows a single solenoid ink jet head 40b. In this embodiment, the momentum transferring device is a solenoid actuator 44b. The electrical impulse from the power source 32 activates a coil 50 that generates a magnetic field, causing the solenoid actuator 44b to draw into the coil 50. A flexible film 48b may be provided near the entry of the chamber 42b of the ink jet head 40b to assist in controlling the flow of composition through the flow path 16b and the chamber 42b until it reaches the orifice 46b. When the solenoid actuator 44b releases from the coil 50, the solenoid actuator 44b assists in forcing the composition out of the orifice 46b.

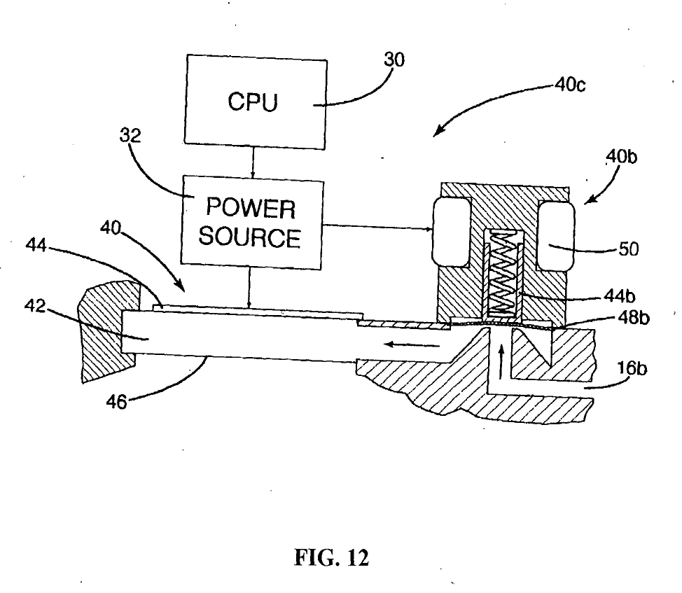

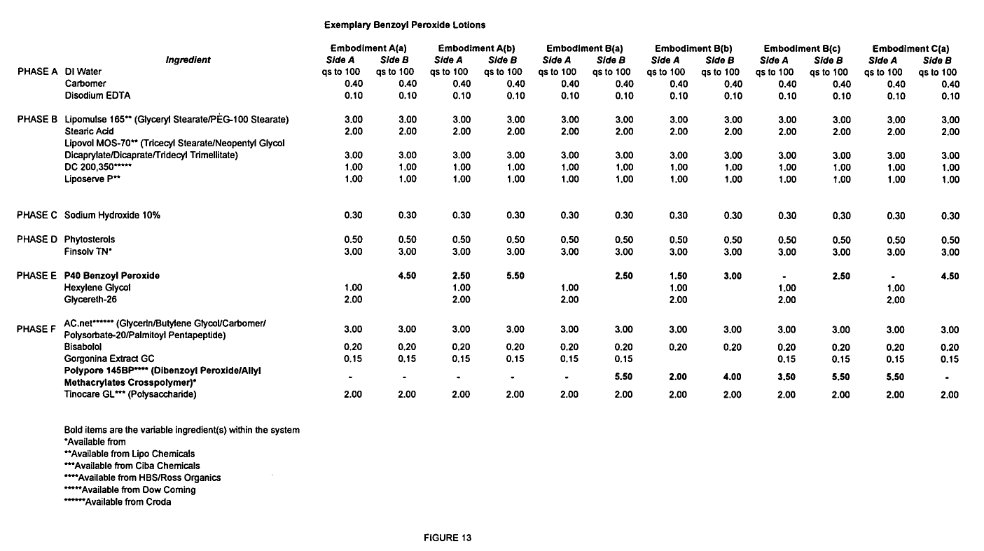

FIG. 12 shows a dual valve solenoid-piezo embodiment of an ink jet head 40c. In this embodiment, a piezoelectric ink jet head 40 is used in combination with a solenoid ink jet head 40b. The composition flows into the solenoid ink jet head 40b and then into the piezoelectric ink jet head 40 for final momentum out of the orifice 46. Similarly, other multi valve ink jet systems can be employed for the present invention. Any combination of thermal, piezo, solenoid, and magneto-restrictive alloy or other suitable materials may be incorporated into the ink jet head.

In various embodiments, the systems can be connected to a stand alone or remote computer or other control system as desired. Formula information may be stored in the computer's hardware, software, or a website set up for the current dispenser. The computer having the stored formula information may be a colorimeter or a spectrophotometer. The dispenser may have a plug-in for connecting the computer to the dispenser, such as a USB port, serial port, parallel port or other communications port. In operation, the user might choose a given mixture of ingredients using the computer, which would then download the particular formula into a CPU in the dispenser for immediate dispensing. The computer may include a database of pre-created formula or may create the formula in real time through user interaction. The computer may also permit the user to directly enter a formula. The dispenser CPU may include software for converting formulae received from the computer into ink jet head instructions. Alternatively, the computer may convert the formulae into ink jet head instructions that are transmitted to and executed by the dispenser CPU.

The scope of the present systems and methods, etc., includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims.

1. A method of treating acne in a human comprising, providing a dispensing vessel containing one or more anti-acne agents wherein the vessel is configured such that a patient can easily vary the concentration of at least one anti-acne agent in a composition dispensed from the vessel in accord with a desire of the user to treat acne.

2. The method of claim 1 wherein the anti-acne agent is benzoyl peroxide.

3. The method of claim 1 wherein the anti-acne agent is salicylic acid.

4. The method of any one of claims 1 to 3 wherein the method further comprises the user adjusting the concentration of the at least one anti-acne agent in the composition dispensed from the vessel in order to adjust the anti-acne effect of the anti-acne agent.

5. The method of claim 4 wherein the method further comprises simultaneously increasing a concentration of a first anti-acne agent in order to adjust the anti-acne effect of the anti-acne agent while decreasing a concentration of a second anti-acne agent in order to adjust the anti-acne effect of the anti-acne agent.

6. The method of claim 4 wherein the method further comprises simultaneously increasing a concentration of a first anti-acne agent in order to adjust the anti-acne effect of the anti-acne agent and increasing a concentration of a second anti-acne agent in order to adjust the anti-acne effect of the anti-acne agent.

7. The method of claim 4 wherein the method further comprises independently adjusting a concentration of a first anti-acne agent in order to adjust the anti-acne effect of the anti-acne agent and independently adjusting a concentration of a second anti-acne agent in order to adjust the anti-acne effect of the anti-acne agent.

8. The method of claim 4 wherein the first anti-acne agent is benzoyl peroxide and the second anti-acne agent is salicylic acid.

9. The method of claim 8 further comprising adjusting the concentration of the benzoyl peroxide between about 0% and 20% and the salicylic acid between about 0% and 10%.

10. The method of claim 8 further comprising adjusting the concentration of the benzoyl peroxide between about 1%and 10% and the salicylic acid between about 1%and 5%.

11. The method of claim 8 further comprising adjusting the concentration of the benzoyl peroxide between about 2.5% and 10% and the salicylic acid between about 0.5% and 2%.

12. The method of claim 8 further comprising adjusting the concentration of the benzoyl peroxide such that increased concentrations of the benzoyl peroxide are administered when acne of the user is relatively worse and such that decreased concentrations of the benzoyl peroxide are administered when acne of the user is relatively less, and further comprising adjusting the concentration of the salicylic acid such that increased concentrations of the salicylic acid are administered when acne of the user is relatively worse and such that decreased concentrations of the salicylic acid are administered when acne of the user is relatively less.

13. The method of claim 1 wherein the first anti-acne agent comprises at least one of resorcinol, azelaic acid, retinoic acid, retinol, ascorbic acid, alpha-hydroxy acids, beta-hydroxy acids and the second anti-acne agent comprises at least one of resorcinol, azelaic acid, retinoic acid, retinol, ascorbic acid, alpha-hydroxy acids, beta-hydroxy acids.

14. The method of claim 1 wherein the first anti-acne agent and the second anti-acne agent are selected such that the first anti-acne agent has at least one side effect that is ameliorated by the second anti-acne agent.

15. The method of claim 1 wherein the first anti-acne agent and the second anti-acne agent are selected such that at least one of the first anti-acne agent and the second anti-acne agent enhances the anti-acne effect of the other anti-acne agent.

16. The method of claim 1 wherein the method further comprises administering the first anti-acne agent to the skin of the user during the evening and administering the second anti-acne agent to the skin of the user during the morning, and wherein the method further comprises using at least two dispensing vessels, one for each of the anti-acne agents.

17. The method of claim 4 wherein the method further comprises administering at least one skin care active agent.

18. The method of claim 17 wherein the at least one skin care active agent comprises at least one of a tocotrienol, gorgonian, oleanolic acid, zinc, camellia sinensis, NDGA, bisabolol or allantoin.

19. An anti-acne system comprising at least one dispensing vessel containing one or more anti-acne agents wherein the vessel is configured such that a patient can easily vary the concentration of at least one anti-acne agent in a composition dispensed from the vessel in accord with the desires of the user.

20. The anti-acne system of claim 19 wherein the anti-acne agent is benzoyl peroxide.

21. The anti-acne system of claim 19 wherein the anti-acne agent is salicylic acid.

22. The anti-acne system of claim 19 wherein the anti-acne system further comprises at least two anti-acne agents and the system is configured such that the user can simultaneously increase a concentration of a first anti-acne agent while decreasing a concentration of a second anti-acne agent.

23. The anti-acne system of claim 19 wherein the anti-acne system further comprises at least two anti-acne agents and the system is configured such that the user can simultaneously increase a concentration of a first anti-acne agent and increase a concentration of a second anti-acne agent.

24. The anti-acne system of claim 19 wherein the anti-acne system further comprises at least two anti-acne agents and the system is configured such that the user can independently adjust a concentration of a first anti-acne agent and independently adjust a concentration of a second anti-acne agent.

25. The anti-acne system of any one of claims 19 to 24 wherein the dispensing vessel comprises at least a first compartment comprising a first sub-composition comprising the first anti-acne agent at a first concentration and a second compartment comprising a second sub-composition lacking the first anti-acne agent or comprising first anti-acne agent at a second, lower concentration, wherein the dispensing vessel is configured such that the concentration of the at least one anti-acne agent is increased or decreased by varying the relative amount of the first sub-composition and the second sub-composition dispensed from the dispensing vessel.

26. The anti-acne system of claim 25 further comprising at least a third compartment comprising a third sub-composition, wherein the dispensing vessel is configured such that the components of a composition dispensed from the dispensing vessel is varied by varying the relative amount of the first sub-composition, the second sub-composition and the third sub-composition dispensed from the dispensing vessel.

27. The anti-acne system of claim 26 further comprising at least two different anti-acne compositions each contained in different compartments.

28. The anti-acne system of claim 27 further comprising at least three different anti-acne compositions each contained in different compartments.

29. The anti-acne system of claim 19 wherein the first anti-acne agent is benzoyl peroxide and the second anti-acne agent is salicylic acid.

30. The anti-acne system of claim 29 wherein the dispensing vessel is configured such that the concentration of the benzoyl peroxide can be adjusted between about 0% and 20% and the concentration of the salicylic acid can be adjusted between about 0% and 10%.

31. The anti-acne system of claim 29 wherein the dispensing vessel is configured such that the concentration of the benzoyl peroxide can be adjusted between about 1%and 10% and the concentration of the salicylic acid can be adjusted between about 1%and 5%.

32. The anti-acne system of claim 29 wherein the dispensing vessel is configured such that the concentration of the benzoyl peroxide can be adjusted between about 2.5% and 10% and the concentration of the salicylic acid can be adjusted between about 0.5% and 2%.

33. The anti-acne system of claim 29 wherein the dispensing vessel is configured such that the concentration of the benzoyl peroxide can be adjusted such that increased concentrations of the benzoyl peroxide are dispensed when acne of the user is relatively worse and such that decreased concentrations of the benzoyl peroxide are dispensed when acne of the user is relatively less, and such that increased concentrations of the salicylic acid are dispensed when acne of the user is relatively worse and such that decreased concentrations of the salicylic acid are dispensed when acne of the user is relatively less.

34. The anti-acne system of claim 19 wherein the first anti-acne agent comprises at least one of resorcinol, azelaic acid, retinoic acid, retinol, ascorbic acid, alpha-hydroxy acids, beta-hydroxy acids and the second anti-acne agent comprises at least one of resorcinol, azelaic acid, retinoic acid, retinol, ascorbic acid, alpha-hydroxy acids, beta-hydroxy acids.

35. The anti-acne system of claim 19 wherein the first anti-acne agent has at least one side effect that is ameliorated by the second anti-acne agent.

36. The anti-acne system of claim 19 wherein at least one of the first anti-acne agent and the second anti-acne agent enhances the anti-acne effect of the other anti-acne agent.

37. The anti-acne system of claim 19 wherein the anti-acne system is configured such that the first anti-acne agent can be administered to the skin of the user during the evening and such that the second anti-acne agent can be administered to the skin of the user during the morning.

38. The anti-acne system of claim 19 wherein the first anti-acne agent is an oxidizing agent and the second anti-acne agent is an antioxidant.

39. The anti-acne system of claim 19 wherein the anti-acne system further comprises administering at least one skin care active agent.

40. The anti-acne system of claim 19 wherein the at least one skin care active agent comprises at least one of a tocotrienol, gorgonian, oleanolic acid, zinc, camellia sinensis, NDGA, bisabolol or allantoin.

41. The anti-acne system of claim 19 wherein the anti-acne system further comprises at least two dispensing vessels.

42. A method of manufacturing a medicament able to reduce symptoms associated with acne in a human patient, comprising variably combining a pharmaceutically effective amount of at least one anti-acne agent and at least one carrier, adjuvant, excipient, potentiator or skin care active agent to provide a user-variable concentration of the anti-acne agent in an anti-acne composition comprising the at least one anti-acne agent and the at least one carrier, adjuvant, excipient, potentiator or skin care active agent.

43. The method of claim 42 wherein the method further comprises variably combining at least one additional anti-acne agent in the composition.

44. The method of claim 42 comprising variably combining a pharmaceutically effective amount of at least one second anti-acne agent and at least one carrier, adjuvant, excipient, potentiator or skin care active agent to provide a user-variable concentration of the second anti-acne agent in a second anti-acne composition comprising the second anti-acne agent and the at least one carrier, adjuvant, excipient, potentiator or skin care active agent.

45. The method of any one of claims 42 to 44 wherein the method further comprises placing at least one of the compositions in the hand of a user such that the user can administer the at least one of the compositions to skin of the user.

46. A kit for providing compositions able to reduce symptoms associated with acne in a human patient, comprising at least one dispensing vessel according to any one of claims 19 to 24 and instructions for use according to the method of any one of claims 1 to 3.

47. The kit of claim 46 further comprising labeling approved by a governmental agency.
